(19) United States
(12) Patent Application Publication
(10) Pub. No.: US 2008/0045609 A1
(43) Pub. Date: Feb. 21, 2008

(54) METHODS AND THERAPEUTICS TO FACILITATE LIVER REPAIR

(76) Inventor: Dianna Louise DeVore, San Francisco, CA (US)

Correspondence Address: Dianna L DeVore;Jiva Biosciences, Inc., 1001 Mariposa Street, San Francisco, CA, 94107, US

(21) Appl. No.: 11/685,022

(22) Filed: Mar. 12, 2007

Related U.S. Application Data
(60) Provisional application No. 60/781,878, filed on Mar. 12, 2006.

(51) Int. Cl. A61K 35/00; A61P 1/16

(52) U.S. Cl. 514/789

(57) ABSTRACT

The present invention provides compositions, formulations and methods for treating liver diseases related to tissue inflammation and progressive fibrosis, e.g., progressive liver fibrosis following either chronic or acute injury. The compositions of the invention provide the chronic use of therapeutic agents to effect liver repair.

### FIELD OF THE INVENTION

The invention relates generally to treating liver damage by administering therapeutic agents to suppress inflammation and fibrosis and to enhance endogenous repair mechanisms.

### BACKGROUND OF THE INVENTION

Cirrhosis of the liver is a progressive disease of the liver characterized by diffuse damage to hepatic parenchymal cells with nodular regeneration, fibrosis and disturbance of normal architecture. It is associated with failure of hepatic cell function and interference with blood flow and can lead to total hepatic failure and hepatocellular carcinoma (HCC). There are a number of agents that cause hepatocellular injury including alcohol, the hepatitis viruses, various drugs and iron overload (hemochromatosis) amongst others. Exposure to these agents promotes a cascade of inflammatory events that, given repeated exposure, can result in the development of chronic disease including progressive fibrosis and cirrhosis.

The major causes of liver fibrosis in developed nations are viral infection, alcoholic liver disease and non-alcoholic steatohepatitis (NASH). NASH is associated with metabolic syndromes characterized by obesity and insulin resistance, and the prevalence of liver fibrosis due to NASH is predicted to increase steadily with the rise in levels of obesity and associated insulin resistance. As many as 5% of all people in the U.S. are currently predicted to be affected to some degree by NASH, yet all reports to date on the use of treatments for NASH (such as use of TGF-β inhibitors) are anecdotal. NASH has recently been named a new priority area of study and intervention by the National Institutes of Health (NIH) in the U.S.

New therapeutic treatments to prevent or cure inflammation and fibrosis associated conditions, such as those identified above, are needed as the current available therapeutics are inadequate, and these diseases have significant unmet clinical need.

### SUMMARY OF THE INVENTION

The present invention provides compositions, formulations and methods for treating liver diseases related to tissue inflammation and progressive fibrosis, e.g., progressive liver fibrosis following either chronic or acute injury. The compositions of the invention provide the sequential use of therapeutic agents to effect liver repair. Each of these therapeutic agents is characterized by: 1) anti-inflammatory activity; 2) anti-fibrotic activity; and, optionally, 3) the ability to modulate cell proliferation and/or tissue regeneration.

The methods generally comprise administering to an individual in need thereof a pharmaceutical formulation comprising an effective amount of one or more of these therapeutic agents to treat progressive fibrosis in a tissue following injury. The dosage regime is determined by measurement of indices indicative of liver function, and multiple administrations are used to provide the best outcome for the patients. The prevention of both inflammation and fibrosis effected by these agents enhances the environment for regeneration and prevents progressive injury in the organ, e.g., prevents damage caused by viral infection.

A key aspect of the invention is a method of treatment. The method comprises measurement of a liver function parameter of a patient. That parameter can be a parameter including but not limited to blood scores of fibrosis; hyaluronate dosage; prothrombin time; α-2 macroglobulinemia dosages; cytokine levels; expression of TNF-α receptors; and liver histology. For example, blood scores of fibrosis include measurement of ALT and AST (aminotransferases enzymes), alkaline phosphatase, direct and total bilirubin, and albumin. Area of fibrosis can be determined using image analysis such as magnetic resonance imaging (MRI) or positron emission tomography (PET) imaging. Changes in liver histology are determined through liver biopsy.

After allowing for the therapeutic agent to have a desired effect, the liver function parameter or parameters that were initially measured are remeasured. The steps of measuring, administering and remeasuring can be repeated any number of times over a desired period of time. Thus, the series of steps can be repeated 1, 2, 3, 4, 5 . . . 25 times or more over a period of days, weeks, months or years. By repeating the steps a plurality of times a beneficial therapeutic result maybe obtained and that result can be objectively measured by comparing the measurements of one or more of the liver function parameters.

In one embodiment, the invention provides a method of treatment, comprising the steps of:

(a) measuring a liver function parameter of a patient to determine a need for administration of the therapeutic agents;

(b) administering to a patient a formulation comprising one or a combination of therapeutic agents;

(c) remeasuring the parameter of the patient measured in (a) after administration of the therapeutic agents; and

(d) readministering the formulation to the patient.

The administration in step (b) and the readministration in step (d) may include a single dosage or multiple dosages of the therapeutic agents as determined by a medical practitioner.

In a specific embodiment, the liver function parameters measured are chosen from the area of fibrosis; changes by blood scores of fibrosis; hyaluronate dosage; prothrombin time; α-2 macroglobulinemia dosages; cytokine levels; expression of TNF-α receptors; and liver histology.

In one specific aspect of this embodiment, the area of fibrosis is determined by image analysis. This image analysis may be undertaken using multiple different technologies used in the medical imaging field, including magnetic resonance imaging (MRI) or Positron Emission Tomography (PET).

In another specific aspect of this embodiment, the measurement of blood scores fibrosis are selected from changes in ALT, AST, alkaline phosphatase, direct and total bilirubin, and albumin.

In yet another specific aspect of this embodiment changes in liver histology are determined through liver biopsy.

In a preferred embodiment, the invention further provides:

(e) adjusting the amount of formulation readministered in (d) based on an analysis of results obtained in remeasuring in (c).

In a specific embodiment, the invention further provides the step of:

(f) repeating (a)-(e) a plurality of times.

In a specific aspect of this embodiment, steps (a)-(e) are repeated until a measured parameter is improved by 10% or more relative to a level measured in the first step (a).

In a specific embodiment, the agents for use in the present invention are administered through parenteral routes, such as intravenous administration or intraportal injection.

In another embodiment, where one or more agents are administered, they are administered separately. One example would be oral administration of an agent such as an ACE inhibitor and local injection of a therapeutic peptide into the damaged region of the liver. The agents can be administered sequentially or simultaneously to aid in the regenerative process.

The antifibrotic activity of the agents can be mediated by physiological responses including, but not limited to, a decrease in fibroblast activation, a decrease in fibroblast differentiation, a decrease in collagen synthesis and/or deposition, or an increase in matrix metalloproteinase (MMP) expression or activity. The anti-inflammatory effect can be mediated, for example, through a decrease in proinflammatory cytokines, an increase in anti-inflammatory cytokines, or prevention of activation of various cells associated with inflammation, such as mast cells, neutrophils and the like. Modulation of cell proliferation and/or regeneration can include the promotion of cell division within the damaged tissue to replace the areas of damage, recruitment of endogenous cells outside the damaged tissue to the area of injury to improve repair of the tissue, or suppression of cell proliferation and/or activation within the tissue of those cells responsible for activities that promote the tissue damage.

The effect of the therapeutic agents on these respective physiological processes may be direct or indirect. The present invention overcomes shortcomings of the prior art by providing improved methods for providing therapeutic agents with the ability to reduce both fibrosis and inflammation in tissues in need of repair as disclosed herein.

The present invention provides methods of use of either one or a combination of therapeutic agents to decrease organ damage and enhance regeneration upon administration to a patient in need of tissue repair. It is preferable that the therapeutic agents exert their effect primarily in the environment of the damaged tissue, and have little significant effect on normal tissues. For example, the therapeutic peptide relaxin only reduces collagen synthesis and accumulation when stimulated by a number of factors in the damaged tissue. It has no significant effect on collagen synthesis or secretion in normal tissue.

Modes of administration, amounts of therapeutic agents administered, and specific formulations for use in the methods of the present invention, are discussed below.

In one specific aspect of the invention, one or a combination of therapeutic agents can be used to treat organ damage following acute injury. The method includes the induction of a protective effect on surrounding tissue and a decrease in the level of tissue remodelling and/or scarring following acute injury. Reduction in scarring will lead to a measurably smaller area of damage following the acute injury as can be shown and measured three months from injury, six months from injury, and twelve months from injury.

In some embodiments, the invention provides methods of treating a fibrotic disorder, comprising administering to a patient in need thereof one or a combination of therapeutic agents in an amount effective to prevent activation of cells involved in the inflammatory response.

In other embodiments, the invention provides methods of treating a fibrotic disorder, comprising administering to a patient in need thereof one or a combination of therapeutic agents in an amount effective to prevent expression and/or activity of proinflammatory cytokines.

In yet other embodiments, the invention provides methods of treating a fibrotic disorder, comprising administering to a patient in need thereof one or a combination of therapeutic agents in an amount effective to promote expression and/or activity of anti-inflammatory cytokines.

In some embodiments, the invention provides methods of treating a fibrotic disorder, comprising administering to a patient in need thereof one or a combination of therapeutic agents in an amount effective to inhibit the differentiation of activated fibroblasts. In a specific embodiment, the therapeutic agents are administered in an amount that decreases production of collagen by fibroblasts.

In some embodiments, the invention provides methods of treating fibrosis and inflammation, comprising administering to a patient in need thereof one or a combination of therapeutic agents in an amount effective to inhibit the proliferation of activated fibroblasts.

In some embodiments, the invention provides methods of treating a fibrotic disorder, comprising administering to a patient in need thereof a pharmaceutical formulation comprising one or a combination of therapeutic agents in an amount effective to antagonize collagen deposition by activated fibroblasts.

In some embodiments, the invention provides methods of treating a fibrotic disorder, comprising administering to a patient in need thereof one or a combination of therapeutic agents in an amount effective to increase collagen degradation via activation of MMPs.

In one specific aspect of the invention, one or a combination of therapeutic agents can be used to treat liver damage from chronic inflammatory disease, such chronic injury due to hypertension, liver inflammation and damage associated with viral infection or alcoholic liver disease, and the like.

In another specific embodiment, one or a combination of therapeutic agents is used to treat liver fibrosis and to prevent the progression to cirrhosis and failure. Forms of hepatic fibrosis amenable to treatment with the present invention include fibrosis caused by alcoholic liver disease, chronic viral infection (e.g., infection with hepatitis B virus (HBV) or hepatitis C virus (HCV)), including co-infection with two or more virus (e.g., HIV and HCV), chronic damage due to drug toxicity, and genetic forms of hepatic fibrosis.

In a specific embodiment, one or a combination of is used to treat liver disease associated with metabolic syndromes, such as non-alcoholic steatotic hepatitis (NASH).

An aspect of the invention is a method of sequentially a patient population suffering from liver fibrosis. The method comprises measuring a liver function parameter of a patient. That parameter can be a parameter including but not limited to blood scores of fibrosis; hyaluronate dosage; prothrombin time; alpha 2 macroglobulinemia dosages; cytokine levels; expression of TNF-α receptors; and liver histology. For example, blood scores of fibrosis include measurement of ALT and AST (aminotransferases enzymes), alkaline phosphatase, direct and total bilirubin, and albumin. Area of fibrosis can be determined using image analysis such as magnetic resonance imaging (MRI) or positron emission tomography (PET) imaging. Changes in liver histology are determined through liver biopsy.

After allowing for the therapeutic agents to have the desired effect, the liver function parameter or parameters that were initially measured are remeasured. The steps of measuring, administering and remeasuring can be repeated any number of times over a desired period of time. Thus, the series of steps can be repeated 1, 2, 3, 4, 5 . . . 25 times or more over a period of days, weeks, months or years. By repeating the steps a plurality of times a beneficial therapeutic result maybe obtained and that result can be objectively measured by comparing the measurements of one or more of the liver function parameters measured.

Therapeutically effective amounts of the therapeutic agent may be delivered to a patient via systemic delivery, separately or in combination. Systemic administration has the advantages of permitting a physician to have greater control over drug administration, including frequency and dosage, without concern as to whether, for example, a locally administered drug is effectively releasing active ingredient or whether contents of an injection remain at the desired site. Systemic delivery includes, but is not limited to, oral delivery, intravenous injection, subcutaneous injection, pulmonary delivery, and delivery via an implanted osmotic pump.

Therapeutically effective amounts of one or a combination of therapeutic agents may also be delivered to a patient via local delivery. Local delivery provides the agonist such as relaxin to the area of damage directly, and so is a more targeted method for reduction of fibrosis in a specific area of injury. Examples of such delivery include, but are not limited to, administration via a catheter (optionally attached to an osmotic pump), direct injection into or near the damaged liver tissue, injection into the pericardium, a depot injection, and the like.

These and other objects, advantages, and features of the invention will become apparent to those persons skilled in the art upon reading the details of the methods and formulations as more fully described below.

### DETAILED DESCRIPTION OF THE INVENTION

Before the present methods, formulations and treatments are described, it is to be understood that this invention is not limited to the particular methodology, products, delivery apparatus and treatments described, as such methods, treatments and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “an agent” refers to one or mixtures of agents, and reference to “the method of administration” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of cell culture, stem cell biology, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol NO. I, Cold Spring Harbor Laboratory (1998); and Harlow and Lane, Antibodies: A Laboratory Manual, Cold Spring Harbor Laboratory; (1988).

Although the present invention is described primarily with reference to liver fibrosis, it is also envisioned relaxin may play a significant role in resolving the fibrotic response following injury of other organ systems (e.g., cardiac or renal). The present invention is intended to cover these uses of therapeutic peptides as well as the hepatic uses emphasized herein.

### DEFINITIONS

As used in the present specification, the following words and phrases are generally intended to have the meanings as set forth below, except to the extent that the context in which they are used indicates otherwise.

The terms “treat,” “treatment” and the like are used herein to generally mean obtaining a desired pharmacological and/or physiological effect. A treatment is an approach for obtaining beneficial or desired clinical results which include but are not limited to treating patients following and/or during either chronic or acute liver injury. The effect may be prophylactic in terms of completely or partially preventing a disease and/or symptom thereof and/or effect of an injury and/or may be therapeutic in terms of a partial or complete cure of the disease, injury and/or adverse effect attributed to the disease or injury. In general, methods of the invention involve treating and/or healing an adverse effect associated with either chronic and/or acute liver injury. “Treatment” as used herein covers any treatment of such a symptom or disease or injury in a mammal, particularly a human, and includes:

(a) preventing or diagnosing the injury and/or disease and/or symptoms in the subject which may be predisposed to the injury and/or disease and/or symptom but has not yet been diagnosed as having it;

(b) inhibiting the injury and/or disease, i.e. arresting it's development; and/or

(c) relieving or healing injury and/or disease and/or it's symptom, i.e. causing healing of the injury and/or regression of the disease and/or the symptoms caused by the injury and/or disease.

Current therapy therefore depends on balancing the effects of multiple drugs to achieve the clinical needs of individual patients, and is plagued by adverse reactions to the drugs used. The treatment may include measuring a parameter of a patient and then administering a formulation. Thereafter, the parameter is remeasured and the formulation readminstered wherein dosing may by adjusted based on effects determined by a differential between the first measuring and the remeasuring after administering formulation. These and other types of treatment will occur to those skilled in the art upon reading this disclosure.

The terms “synergistic”, “synergistic effect” and the like are used interchangeably herein to describe improved treatment effects obtained by combining different formulations with the method of the invention. Although a synergistic effect in some fields means an effect which is more than additive (e.g., one plus one equals three) in the field of treating patients and the like and related injuries and diseases an additive (one plus one equals two) or less than additive (one plus one equals a number greater than 1 but less than 2) effect may be synergistic.

“Therapeutic agent” means any pharmaceutically active peptide, small molecule, nucleotide or ribonucleotide agent, or other biologically active moiety which can exhibit both antifibrotic effects and anti-inflammatory effects upon administration to a patient. Relevant antifibrotic activities of a therapeutic agent include: 1) inhibiting the activation of fibroblasts; 2) inhibiting the proliferation of activated fibroblasts; 3) antagonizing collagen deposition by activated fibroblasts; and 4) increasing collagen degradation via activation of MMPs. The therapeutic agents will also inhibit unwanted inflammation in the following exemplary ways: 1) increase in anti-inflammatory cytokines such as IL-4 and IL-10; 3) decrease in proinflammatory cytokines such as IL1B, CCR2 (MCP1-R), CXCL1 (GRO1), CXCL3 (GRO3), and CCL13 (MCP4), 2) inhibition of platelet activation, 3) inhibition of mast cell degranulation 4) inhibition of neutrophil activation and 5) inhibition of calcium influx.

The therapeutic agents include peptides with specific domains having the desired activities (including monoclonal antibodies), combinations of distinct peptides from a single class or family, combinations of peptides of different classes and families, and combinations of peptides and small molecules. Peptide-based therapeutic agents are referred to herein as “therapeutic peptides”.

Therapeutic agents of the invention specifically include (but are not limited to):
1) Recombinant or synthesized proteins of the IGF-1 superfamily, and more specifically IGF-1 and relaxin or active domains thereof. For IGF-1, the preferred isoform comprises an active IGF-1 E peptide.
2) TGF-β superfamily antagonists, including direct inhibitors of receptors and indirect inhibitors of TGF-β1 signaling activity. These peptides include Activin A inhibitors (e.g., follistatin), ALK1-8 kinase inhibitors (e.g., ALK5 kinase inhibitor), interferons (e.g., interferon alpha and interferon gamma), focal adhesion kinase (FAK kinase) inhibitors, phosphatidylinositol 3-kinase inhibitors (PI 3-kinase), AKT inhibitors.
3) Agents that inhibit the renin-angiotensin system, including but not limited to ACE inhibitors, Angiotensin II Receptor Binding Proteins (ARBs) and any follow on therapeutics based on these activities.
4) Interferons, including interferon-γ and interferon-α.
5) Natriuretic peptides, including both B-type natriuretic peptide and atrial natriuretic peptide.

By a polypeptide demonstrating “functional activity” is meant, a recombinant or synthetic polypeptide capable of displaying one or more known functional activities associated with the native molecule.

The term “suitable control” is used herein to refer to a subject not treated with drug or a subject treated with the same formulation or excipient materials which do not contain the drug. A suitable control may be a subject measured prior to treatment as compared with the same subject after treatment. A suitable control may be an age-matched or otherwise matched subject not treated with drug or treated with placebo.

### The Invention in General

Liver fibrosis, even fairly advanced fibrosis, is known to be reversible, but it may take years for significant regression to be achieved and it is not clear that a fibrotic liver can regress to a normal liver without intervention. The present invention provides therapies to resolve liver fibrosis and aid the regenerative process by using anti-fibrotic and anti-inflammatory agents as an adjunct therapy with cell transplantation. The ability to promote liver repair and reverse the effects of on-going damage will improve the quality of life of millions of people with significant unmet clinical need.

Without being bound to a specific theory, the invention is based, in part, on the novel finding that agents with both anti-inflammatory and antifibrotic activity will enhance the body's own regenerative response. Specifically, the present invention provides compositions, formulations and methods to aid in the efficacy of introduced cells by accelerating the endogenous process of fibrosis resolution in humans, either following cessation of injury or in the context of on-going damage to prevent further disease progression. The ability to promote liver repair through the sequential administration of one or a combination of therapeutic agents will improve the quality of life of millions of people with significant unmet clinical need.

In addition to monotherapies, a specific embodiment of the invention provides use of a combination of therapeutic agents that are working through various pathways to control inflammation and fibrosis. Combined use will provide mechanisms for overcoming the body's ability to compensate through redundant pathways.

In addition to their anti-inflammatory and anti-fibrotic activity, certain therapeutic agents of the invention will also have the ability to modulate cell proliferation, and thus aid even further in the combined use of the agents and the cells. This can either be a promotion of the functional integration of the cells with the patient's tissue, a concurrent stimulation of activity of the endogenous cell populations, or the repression of the proliferation of unwanted cell populations, as with the repression of hepatic stellate cell (HSC) proliferation in the liver by PI3 kinase.

The antifibrotic activity of the agents can be mediated by physiological responses including, but not limited to, a decrease in fibroblast activation, a decrease in fibroblast differentiation, a decrease in HSC activation, a decrease in collagen synthesis and/or deposition, or an increase in matrix metalloproteinase (MMP) expression or activity. The anti-inflammatory effect can be mediated, for example, through a decrease in proinflammatory cytokines, an increase in anti-inflammatory cytokines, or prevention of activation of various cells associated with inflammation, such as mast cells, neutrophils and the like. Modulation of cell proliferation and/or regeneration can include the promotion of cell division within the damaged tissue to replace the areas of damage, recruitment of endogenous cells outside the damaged tissue to the area of injury to improve repair of the tissue, or suppression of cell proliferation and/or activation within the tissue of those cells responsible for activities that promote the tissue damage.

### Therapeutic Agents for Adjunct Therapies

The therapeutic agents of the present invention include peptides and small molecules that modulate various signaling pathways involved in inflammation and/or fibrosis. These can be small molecules designed to impact on intracellular signaling, recombinant versions of naturally occurring peptides, and therapeutic monoclonal antibodies that specifically alter the activity of transmembrane receptors.

The invention thus includes not only the therapeutic agents described below, but also any mimetics, monoclonal antibodies, and the like designed to mimic such activity and/or modulate the same pathways.

Peptides of the Insulin Superfamily

The insulin gene family, comprised of insulin, relaxin, insulin-like growth factors I and II (IGF-I and IGF-II), represents a group of structurally related polypeptides. Although the molecules affect different physiological processes, certain members have both an anti-inflammatory and anti-fibrotic effect when expressed in or administered to damaged tissue.

Relaxin

Relaxin is a hormone of the insulin superfamily produced during pregnancy that facilitates the birth process by causing a softening and lengthening of the cervix and the pubic symphysis. Relaxin works by simultaneously cutting collagen production and increasing collagen breakdown.

Relaxin is a heterodimer protein with a molecular weight of 6 kD. Relaxin is a naturally occurring regulator of collagen turnover, with the ability to both limit collagen production and reorganization, and to stimulate increased collagen degradation (For review, see Samuel C S, Clin Med. Res. 2005 November; 3(4): 241-249). Recombinant human relaxin has been shown to reduce fibrosis associated with organ damage in a variety of animal models, including reversing cardiac and renal fibrosis in a rat model of hypertension (Lekgabe et al., Hypertension. 2005 August; 46(2):412-8). Specifically, recombinant human relaxin has been demonstrated to inhibit collagen deposition by hepatic stellate cells (HSCs) in a rat CCl₄ model of liver damage (Williams E J et al., Gut. 2001 October; 49(4):577-83).

Relaxin is known to act through multiple receptors, including two G-protein coupled receptors (LGR7 and LGR8) and the glucocorticoid receptor. LGR7 and LGR8 are upregulated in activated HSC, and it is believed that these relaxin receptors may be involved in inhibition of sustained HSC activation during scar resolution in the regenerative healing process. (Bennett et al., Ann NY Acad Sci 2005 1041:185-189). Based on in vitro and in vivo studies, it is believed that the majority of the anti-fibrotic activity of relaxin is mediated through its highest affinity receptor, LGR7.

From previous clinical use, relaxin has a proven history of safety and systemic administration has been associated with few adverse events (Seibold J R et al., Ann Intern Med. 2000 Jun. 6; 132(11):871-9).

Insulin Growth Factor-1 (IGF-1)

Insulin-like growth factor 1 (IGF-1) is a peptide hormone produced in numerous tissues particularly by the liver in response to growth hormone stimulation and is an important factor in the regulation of post-natal growth and development. The rationale for using subdomains and specific sub-peptides of IGF-1 to effect tissue repair is based on initial preclinical results that suggest that specific isoforms of IGF-1 can improve heart function and prevent progression to end stage heart failure by: 1) decreasing the inflammatory response following injury in the heart; 2) reducing fibrosis, thus decreasing the ensuing remodeling events; and 3) promoting regeneration of heart tissue following injury by stimulating cell proliferation. These physiological activities are fundamental in nature, and thus the antifibrotic and anti-inflammatory activities of IGF-1 will be efficacious in the liver as well.

The therapeutic IGF-1 peptides may be isoforms that include specific c-terminal peptides (E peptides). These peptides will have biological activity in cell culture, and in vivo using established tissue damage and disease paradigms. A battery of molecular, cellular, structural and functional tests will be employed to determine whether the peptides, delivered systemically or locally, can have the intended effect on liver regenerative parameters.

TGF-β Superfamily Inhibitors

TGF-β1 Inhibitors

Transforming growth factor β1 (TGF-β1) is a member of a large superfamily of pleiotropic cytokines that are involved in many biological activities, including growth, differentiation, migration, cell survival, and adhesion in diseased and normal states. The members of this superfamily fall into two major branches: TGF-β/Activin/Nodal and BMP/GDF (Bone Morphogenetic Protein/Growth and Differentiation Factor). They have very diverse and often complementary functions. Some are expressed only for short periods during embryonic development and/or only in restricted cell types (e.g., anti-Mullerian hormone, AMH, and Inhibin) while others are widespread during embryogenesis and in adult tissues (e.g., TGF-β1 and BMP4). TGF-β1 is a potent regulator in the synthesis of the extracellular matrix (fibrotic factor) and plays a role in wound healing.

TGF-β ligand binding induces receptor complex formation consisting of receptor type II and I, both of which are serine/threonine kinases. The type II receptor phosphorylates and activates the type I receptor within the complex. Seven type I receptors have been identified to date (Activin Receptor-Like Kinases, ALKs 1-7), and five mammalian type II receptors (TβR-II, ActR-II, ActR-IIB, BMPR-II, AMHR-II). The relationship between different TGF-β family ligands and usage of receptor types II and I have been reviewed (Massague J., et al., Cell, 103, 295 (2000)). Downstream signal transduction takes place when the phosphorylated type I receptor phosphorylates transcription factors, R-Smad or Smad substrates for receptors (Smads 1-8). In general ALK-4, -5, -7, corresponding to the TGFβ/Activin/Nodal branch, phosphorylates Smad-2 and -3, while Smad-1, -5, -8 are substrates for ALK-1, -2, -3, and -6 corresponding to the BMP/GDF branch. These phosphorylated Smads then interact with the co-Smad, Smad 4, at high affinity. These Smad complexes accumulate in the nucleus, are required for the assembly of transcriptional apparatus and directly interact with target genes.

Inhibitors of TGF-β1 signaling in particular are a large focus of activity for many companies looking to reverse organ damage. They provide an important component for formulations of the invention alone or in combination with other therapeutic agents. Combinations including TGF-β1 signaling antagonists provide mechanisms for attacking molecular redundancy, in combination with members of the insulin superfamily (e.g., IGF-1 or relaxin) or with antagonists of other members of the TGF-β1 superfamily (e.g., follistatin as an antagonist of Activin A).

Activin A Inhibitors

Follistatin is a peptide inhibitor of Activin A, which is a member of the transforming growth factor-beta superfamily. Activin A is constitutively expressed in hepatocytes and regulates liver mass through tonic inhibition of hepatocyte DNA synthesis. Activin A expression is upregulated in later stages of fibrosis, including hepatic fibrosis (Huang et al., World J Gastroentero 2001 7(1): 37-41), and Activin A has been shown to be produced by activated HSCs in both in vitro and in vivo studies (Patella S et al., Am J Physiol Gastrointest Liver Physiol. 2006, 290(1):G137-44). Physiological effects of follistatin include the prevention of apoptosis stimulated by Activin A and the promotion of tissue regeneration following injury (Kogure et al., Hepatology, 1996 24(2):361-366). Similarly, Follistatin has been shown to inhibit TGF-β-induced secretion of collagen from HSC (Wada W et al., Endocrinology. 145(6):2753-9 (2004)).

Follistatin has been shown to cause a 32% reduction in fibrosis in CCl₄-exposed rats treated with the peptide. During treatment, hepatocyte apoptosis decreased by 87% and the effect was maximal at week 4 of follistatin treatment. These results indicate that follistatin attenuates early events in fibrogenesis by constraining HSC proliferation and inhibiting hepatocyte apoptosis (Patella, supra). Absence of simultaneous upregulation of follistatin gene expression with Activin A upregulation in activated HSCs suggests that HSC-derived Activin A is biologically active and unopposed by follistatin, and that introduced follistatin could be a powerful clinical intervention (Patella, supra).

Follistatin has been shown to actively promote cell replication and increase liver size through physiological mechanisms following partial hepatectomy (Kogure et al., Hepatology 1996, August: 24(2):361-366), and thus may effect regeneration in damaged liver upon clinical administration. Importantly, although follistatin significantly increases liver weight after hepatic resection, it did not accelerate tumor cell growth in either in vitro or in vivo studies (Fuwii M et al., Hepatogastroenterology. 2005 May-June; 52 (63):833-8).

Interferons

Interferon-α

Interferon-α is an important cytokine in the early immune response to viral infection and has both antiproliferative and antiviral properties. Interferon-α has been used at high doses (1 million to 50 million U) for the treatment of malignant disorders and infectious diseases, including malignant melanoma and chronic hepatitis C. Interferon-α, in combination with ribavirin, is the only therapy approved by the Food and Drug Administration for hepatitis C virus infection, which affects 4 million to 5 million persons in the United States and is the most common cause of cirrhosis leading to liver transplantation.

Interferon-γ

The proliferation of fibroblasts and the accumulation of interstitial collagens are the hallmarks of progressive organ fibrosis. In vitro studies have demonstrated that interferon-γ inhibits the proliferation of lung fibroblasts in a dose-dependent manner and reduces the synthesis of protein in fibroblasts. Moreover, in a bleomycin-induced model of lung fibrosis, exogenous interferon-γ down-regulated the transcription of the gene for TGF-β1. This growth factor has been demonstrated to cause severe lung fibrosis in rats with adenovirus vector-mediated overexpression of the cytokine. It also has a major role in collagen synthesis as well as in the proliferation and activation of fibroblasts. In contrast to the immunomodulatory function of TGF-β1, the effects of this growth factor on the regulation of wound healing and fibrosis are mediated by the action of connective-tissue growth factor. A study of various forms of pulmonary fibrosis, including idiopathic pulmonary fibrosis, has indicated that there may be a general impairment of the production of interferon-γ in patients with pulmonary fibrosis. In addition, another study reported that treatment of progressive pulmonary fibrosis with interferon γ-1b was effective in patients who had idiopathic pulmonary fibrosis, scleroderma, or sarcoidosis that was resistant to three months of treatment with high doses of glucocorticoids.

Natriuretic Peptides

B-type natriuretic peptide and atrial natriuretic peptide are peptide hormones released in response to myocyte stretch. B-type natriuretic peptide is released primarily by ventricular myocytes in the form of the active hormone and an inactive N-terminal fragment, whereas atrial natriuretic peptide and its inactive N-terminal fragment are released primarily by the atria. Both of these hormones augment urinary volume and urinary sodium excretion, relax vascular smooth muscle, and inhibit the sympathetic nervous system and the renin-angiotensin-aldosterone system.

Renin-Angiotensin System Inhibitors

The renin-angiotensin-aldosterone system plays an important role in regulating physiological characteristics such as blood volume, arterial pressure, and cardiac and vascular function. While the pathways for the renin-angiotensin system have been found in a number of tissues, the most important site for renin release is the kidney. Sympathetic stimulation (acting via β_{I}-adrenoceptors), renal artery hypotension, and decreased sodium delivery to the distal tubules stimulate the release of renin by the kidney. Renin is an enzyme that acts upon a circulating substrate, angiotensinogen, that undergoes proteolytic cleavage to from the decapeptide angiotensin I. Vascular endothelium, particularly in the lungs, has an enzyme, angiotensin converting enzyme (ACE), that cleaves off two amino acids to form the octapeptide, angiotensin II (AII).

ACE Inhibitors

ACE inhibitors produce vasodilation by inhibiting the formation of angiotensin II. This vasoconstrictor is formed by the proteolytic action of renin (released by the kidneys) acting on circulating angiotensinogen to form angiotensin 1. Angiotensin I is then converted to angiotensin 11 by angiotensin converting enzyme. ACE also breaks down bradykinin (a vasodilator substance). Therefore, ACE inhibitors, by blocking the breakdown of bradykinin, increase bradykinin levels, which can contribute to the vasodilator action of ACE inhibitors.

Importantly for use in methods of the invention, ACE inhibitors are known to exhibit anti-fibrotic effects, and have been shown to inhibit cardiac and vascular remodeling associated with chronic hypertension, heart failure, and myocardial infarction. These effects should be mirrored in the use of ACE inhibitors for treatment of liver failure

Angiotensin II Receptor Blockers (ARBs)

These drugs have very similar effects to angiotensin converting enzyme (ACE) inhibitors and are used for the same indications (hypertension, heart failure, post-myocardial infarction). Their mechanism of action, however, is very different from ACE inhibitors, which inhibit the formation of angiotensin II. ARBs are receptor antagonists that block type 1 angiotensin II (AT₁) receptors on bloods vessels and other tissues such as the heart. These receptors are coupled to the Gq-protein and IP₃ signal transduction pathway that stimulates vascular smooth muscle contraction. Because ARBs do not inhibit ACE, they do not cause an increase in bradykinin, which contributes to the vasodilation produced by ACE inhibitors. Similar to ACE Inhibitors, ARBs are known to exhibit anti-fibrotic effects, and have been shown to inhibit cardiac and vascular remodeling.

### Compositions and Administration

Pharmaceutical formulations of therapeutic agents are prepared for storage by mixing the compounds having the desired degree of purity with optional physiologically acceptable carriers, excipients, or stabilizers (Remington's Pharmaceutical Sciences, supra), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The therapeutic agent to be used for in vivo administration must be sterile. This is readily accomplished by filtration through sterile filtration membranes, prior to or following lyophilization and reconstitution. The therapeutic agent ordinarily will be stored in lyophilized form or in solution.

Therapeutic agent compositions generally are placed into a container having a sterile access port, for example, an intravenous solution bag or vial having a stopper bgpierceable by a hypodermic injection needle. The route of therapeutic agent administration is in accord with known methods.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al., Biopolymers 22:547-556 (1983)), poly(2-hydroxyethyl-methacrylate) (Langer et al., J. Biomed. Mater. Res. 15:167-277 (1981) and Langer, Chem. Tech. 12:98-105 (1982)), ethylene vinyl acetate (Langer et al., supra) or poly-D-(−)-3-hydroxybutyric acid (EP 133,988). Sustained-release therapeutic agent compositions also include liposomally entrapped therapeutic agent. Liposomes containing therapeutic agent are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal therapeutic agent therapy.

An “effective amount” of therapeutic agent to be employed therapeutically will depend, for example, upon the route of administration, and the condition of the patient. Accordingly, it will be necessary for the therapist to titer the dosage and modify the route of administration as required to obtain the optimal therapeutic effect. Typically, the clinician will administer the therapeutic agent until a dosage is reached that achieves the desired effect. The progress of this therapy is easily monitored by conventional assays, and will depend upon the indication treated. For example, with liver fibrosis as an indication, an increase in liver function parameters or a decrease in the fibrotic tissue of the liver (as evidenced by MRI) can be used to monitor the appropriate dosing and desired effect on the patient.

In the treatment of organ damage by compositions of the invention, the therapeutic agent composition will be formulated, dosed, and administered in a fashion consistent with good medical practice. Factors for consideration in this context include the particular patient being treated, the clinical condition of the individual patient, the site of delivery of the therapeutic agent, the particular type of therapeutic agents used an their potential interactions or contraindications, the method of administration, the scheduling of administration, and other factors known to medical practitioners. The “therapeutically effective amount” of therapeutic agent to be administered will be governed by such considerations, and is the minimum amount necessary to ameliorate, or treat the liver failure. Such amount is preferably below the amount that is toxic to the host or renders the host significantly more susceptible to infections.

For small molecule therapeutic agents, they will typically be administered using oral formulations. Typical oral formulations include tablets, capsules, syrups, elixirs and suspensions.

Pharmaceutically acceptable carriers for use in the formulations described above are exemplified by: sugars such as lactose, sucrose, mannitol and sorbitol, starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone, polyvinyl alcohol; stearic acid; alkaline earth metal stearates such as magnesium stearate and calcium stearate, stearic acid, vegetable oils such as peanut oil, cottonseed oil, sesame oil, olive oil and corn oil; non-ionic, cationic and anionic surfactants; ethylene glycol polymers; betacyclodextrin; fatty alcohols and hydrolyzed cereal solids; as well as other nontoxic compatible fillers, binders, disintegrants, buffers, preservatives, antioxidants, lubricants, flavoring agents, and the like commonly used in pharmaceutical formulations.

Therapeutic agents such as peptides will be administered using other delivery mechanisms, as they are generally not orally bioavailable. Examples of parenteral administration include subcutaneous, intramuscular, intravenous, intraarterial, and intraperitoneal administration, or by sustained release systems as noted below. Subcutaneous and intravenous injection or infusion is preferred. Also, as advances in the administration of protein therapeutics are made, it will be well within the skill on one in the art to adapt such advances to the therapeutic agents in the delivery of the compositions, formulations, and performance of the methods of the invention as described herein

As a general proposition, the total pharmaceutically effective amount of therapeutic agent administered parenterally per dose will be in the range of about 1 μg/kg/day to 10 mg/kg/day of patient body weight, although, as noted above, this will be subject to a great deal of therapeutic discretion. More preferably, this dose is at least 10 μg/kg/day, and most preferably for humans between about 25-50 μg/kg/day. If given continuously, the therapeutic agent is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. Preferably, in human patients, a pharmaceutically effective amount of the therapeutic agent administered parenterally per dose will be in the range of about 10 to 100 micrograms per kilogram of patient body weight per day.

Practitioners devising doses of therapeutic agents should take into account the known side effects and contraindications of the peptides. As noted above, however, these suggested amounts of therapeutic agent are subject to a great deal of therapeutic discretion. The key factor in selecting an appropriate dose and scheduling is the result obtained, as indicated above.

Since the present invention relates to treatment of fibrosis with combinations of therapeutic agents which may be administered separately, the invention also includes kits comprising separate pharmaceutical compositions for administration to a patient. The kit would comprise two or more separate units: for example, a small molecule composition such as an ACE inhibitor or ARB pharmaceutical composition as one component of the kit, and a relaxin or follistatin peptide pharmaceutical composition as a second component of the kit. The kit form is particularly advantageous when the separate components must be administered in different dosage forms (e.g. oral and parenteral) or are administered at different dosage intervals.

Therapeutic peptides may be administered as a polypeptide, or as a polynucleotide comprising a sequence which encodes relaxin. Peptides may be isolated from natural sources, may be chemically or enzymatically synthesized, or produced using standard recombinant techniques known in the art. For example, methods of making recombinant relaxin are found in various publications, including, e.g., U.S. Pat. Nos. 4,835,251; 5,326,694; 5,320,953; 5,464,756; and 5,759,807.

In general, a daily dose of a therapeutic agent may be from about 0.1 to 500 μg/kg of body weight per day, from about 6.0 to 200 μg/kg, or from about 12 to 100 μg/kg. In some embodiments, it is desirable to obtain a serum concentration of therapeutic peptide at or above about 1.0 ng/ml, from about 0.5 to about 50 ng/ml, from about 1 to about 20 ng/ml. For administration to a 70 kg person, a dosage may be in a range of from about 2 μg to about 2 mg per day, from about 10 μg to 500 μg per day, or from about 50 μg to about 100 μg per day. The amount of therapeutic agent administered will, of course, be dependent on the subject and the severity of the affliction, the manner and schedule of administration and the judgment of the prescribing physician. The amount administered in each subsequent dose may be adjusted based on effects, if any, on a measured parameter. One or more parameters may be measured prior to initial dosing and remeasured prior to each or some subsequent dosing. This allows dosing to be adjusted as desired and the adjustment, if desired, to be based on the effects on one or more measured parameters.

In employing therapeutic peptide for treatment of diseases relating to liver fibrosis any pharmaceutically acceptable mode of administration can be used. Therapeutic peptide can be administered either alone or in combination with other pharmaceutically acceptable excipients, including solid, semi-solid, liquid or aerosol dosage forms, such as, for example, tablets, capsules, powders, liquids, gels, suspensions, suppositories, aerosols or the like. Therapeutic peptide can also be administered in sustained or controlled release dosage forms (e.g., employing a slow release bioerodible delivery system), including depot injections, osmotic pumps (such as the Alzet implant made by Alza), pills, transdermal and transcutaneous (including electrotransport) patches, and the like, for prolonged administration at a predetermined rate, preferably in unit dosage forms suitable for single administration of precise dosages.

The compositions will typically include a conventional injectable pharmaceutical carrier or excipient along with the therapeutic peptide. In addition, these compositions may include other active agents (e.g., other angiogenic agents, other vasodilation-promoting agents), carriers, adjuvants, etc. Generally, depending on the intended mode of administration, the pharmaceutically acceptable composition will contain about 0.1% to 90%, about 0.5% to 50%, or about 1% to about 25%, by weight of the active component which may be stem cells and/or therapeutic peptide, the remainder being suitable pharmaceutical excipients, carriers, etc. Actual methods of preparing such dosage forms are known, or will be apparent, to those skilled in this art; for example, see Remington's Pharmaceutical Sciences, Mack Publishing Company, Easton, Pa., 15th Edition, 1995.

Parenteral administration is generally characterized by injection, either subcutaneously, intradermally, intramuscularly or intravenously, or subcutaneously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, and the like.

The percentage of therapeutic agent contained in such parenteral compositions is highly dependent on the specific nature thereof, as well as the needs of the subject. However, percentages of active ingredient of 0.01% to 10% in solution are employable, and will be higher if the composition is a solid which will be subsequently diluted to the above percentages. In general, the composition will comprise 0.2-2% of stem cells and/or therapeutic agent in solution.

Parenteral administration may employ the implantation of a slow-release or sustained-release system, such that a constant level of dosage is maintained. Various matrices (e.g., polymers, hydrophilic gels, and the like) for controlling the sustained release, and for progressively diminishing the rate of release of active agents such as therapeutic peptides are known in the art. See, U.S. Pat. No. 3,845,770 (describing elementary osmotic pumps); U.S. Pat. Nos. 3,995,651, 4,034,756 and 4,111,202 (describing miniature osmotic pumps); U.S. Pat. Nos. 4,320,759 and 4,449,983 (describing multichamber osmotic systems referred to as push-pull and push-melt osmotic pumps); and U.S. Pat. No. 5,023,088 (describing osmotic pumps patterned for the sequentially timed dispensing of various dosage units).

Drug release devices suitable for use in administering the therapeutic agent may be based on any of a variety of modes of operation. For example, the drug release device can be based upon a diffusive system, a convective system, or an erodible system (e.g., an erosion-based system). For example, the drug release device can be an osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump, are also suitable for use with the present invention. Examples of such devices include those described in, for example, U.S. Pat. Nos. 4,692,147; 4,360,019; 4,487,603; 4,360,019; 4,725,852, and the like. In general, the present treatment methods can be accomplished using any of a variety of refillable, non-exchangeable pump systems. Osmotic pumps have been amply described in the literature. See, e.g., WO 97/27840; and U.S. Pat. Nos. 5,985,305 and 5,728,396. Implanted pumps can be slowly release therapeutic agent and the rate of release can be adjusted based on effects on measured parameters.

The therapeutic agent may be administered over a period of hours, days, weeks, or months, depending on several factors, including the severity of the disease being treated, whether a recurrence of the disease is considered likely, etc. The administration may be constant, e.g., constant infusion over a period of hours, days, weeks, months, etc. Alternatively, the administration may be intermittent, e.g., therapeutic agent may be administered once a day over a period of days, once an hour over a period of hours, or any other such schedule as deemed suitable.

Formulations of therapeutic agent may also be administered to the respiratory tract as a nasal or pulmonary inhalation aerosol or solution for a nebulizer, or as a microfine powder for insufflation, alone or in combination with an inert carrier such as lactose, or with other pharmaceutically acceptable excipients. In such a case, the particles of the formulation may advantageously have diameters of less than 50 micrometers, preferably less than 10 micrometers. For pulmonary delivery the particle sizing may be 2 to 5 micrometers.

The therapeutic agent may be used in conjunction with other therapeutics, and in particular when treating viral infection or metabolic disorder. Standard of care generally includes antivirals for patients with viral hepatitis or insulin for patients with Type II diabetes and metabolic disorders. Current therapy therefore depends on balancing the effects of multiple drugs to achieve the clinical needs of individual patients, and is plagued by adverse reactions to the drugs used.

The therapeutic agent may be administered to an individual in the form of a polynucleotide comprising a nucleotide sequence which encodes the protein of interest. Gene therapy vehicles for delivery of constructs including a coding sequence of a polynucleotide of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

Recombinant retroviruses which are constructed to carry or express a selected nucleic acid molecule of interest can be designed to deliver sequences that the therapeutic peptide. Retrovirus vectors that can be employed include those described in EP 415 731; WO 90/07936; WO 94/03622; WO 93/25698; WO 93/25234; U.S. Pat. No. 5,219,740; WO 93/11230; WO 93/10218; Vile and Hart (1993) Cancer Res. 53:3860-3864; Vile and Hart (1993) Cancer Res. 53:962-967; Ram et al. (1993) Cancer Res. 53:83-88; Takamiya et al. (1992) J. Neurosci. Res. 33:493-503; Baba et al. (1993) J. Neurosurg. 79:729-735; U.S. Pat. No. 4,777,127; and EP 345,242.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Packaging cell lines may be made from human (such as HT1080 cells) or mink parent cell lines, thereby allowing production of recombinant retroviruses that can survive inactivation in human serum.

Gene delivery vehicles of the present invention can also employ parvovirus such as adeno-associated virus (AAV) vectors. Representative examples include the AAV vectors disclosed by Srivastava in WO 93/09239, Samulski et al. (1989) J. Vir. 63:3822-3828; Mendelson et al. (1988) Virol. 166:154-165; and Flotte et al. (1993) Proc. Natl. Acad. Sci. USA 90:10613-10617.

Also of interest are adenoviral vectors, e.g., those described by Berkner, Biotechniques (1988) 6:616-627; Rosenfeld et al. (1991) Science 252:431-434; WO 93/19191; Kolls et al. (1994) Proc. Natl. Acad. Sci. USA 91:215-219; Kass-Eisler et al. (1993) Proc. Natl. Acad. Sci. USA 90:11498-11502; WO 94/12649, WO 93/03769; WO 93/19191; WO 94/28938; WO 95/11984 and WO 95/00655.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell Biol. 14:2411-2418, and in Woffendin (1994) Proc. Natl. Acad. Sci. 91:1581-1585.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 524 968.

Further non-viral delivery suitable for use includes mechanical delivery systems such as the approach described in Woffendin et al. (1994) Proc. Natl. Acad. Sci. USA 91:11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

For administration, one or more of the therapeutic agents in a formulation may be complexed or bound to a polymer to increase its circulatory half-life. Examples of polyethylene polyols and polyoxyethylene polyols useful for this purpose include polyoxyethylene glycerol, polyethylene glycol, polyoxyethylene sorbitol, polyoxyethylene glucose, or the like. The glycerol backbone of polyoxyethylene glycerol is the same backbone occurring in, for example, animals and humans in mono-, di-, and triglycerides.

The polymer need not have any particular molecular weight, but it is preferred that the molecular weight be between about 3500 and 100,000, more preferably between 5000 and 40,000. Preferably the PEG homopolymer is unsubstituted, but it may also be substituted at one end with an alkyl group. Preferably, the alkyl group is a C1-C4 alkyl group, and most preferably a methyl group. Most preferably, the polymer is an unsubstituted homopolymer of PEG, a monomethyl-substituted homopolymer of PEG (mPEG), or polyoxyethylene glycerol (POG) and has a molecular weight of about 5000 to 40,000.

The therapeutic agents can optionally be covalently bonded via one or more of the amino acid residues of the therapeutic agent to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the therapeutic agent. It will be understood, however, that the type and amount of the reactive group chosen, as well as the type of polymer employed, to obtain optimum results, will depend on the particular therapeutic agent employed to avoid having the reactive group react with too many particularly active groups on the therapeutic agent. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

While the residues may be any reactive amino acids on the protein, such as one or two cysteines or the N-terminal amino acid group, preferably the reactive amino acid is lysine, which is linked to the reactive group of the activated polymer through its free epsilon-amino group, or glutamic or aspartic acid, which is linked to the polymer through an amide bond.

The covalent modification reaction may take place by any appropriate method generally used for reacting biologically active materials with inert polymers, preferably at about pH 5-9, more preferably 7-9 if the reactive groups on the therapeutic agent are lysine groups. Generally, the process involves preparing an activated polymer (with at least one terminal hydroxyl group), preparing an active substrate from this polymer, and thereafter reacting the therapeutic agent with the active substrate to produce the therapeutic agent suitable for formulation. The above modification reaction can be performed by several methods, which may involve one or more steps. Examples of modifying agents that can be used to produce the activated polymer in a one-step reaction include cyanuric acid chloride (2,4,6-trichloro-S-triazine) and cyanuric acid fluoride.

In one embodiment the modification reaction takes place in two steps wherein the polymer is reacted first with an acid anhydride such as succinic or glutaric anhydride to form a carboxylic acid, and the carboxylic acid is then reacted with a compound capable of reacting with the carboxylic acid to form an activated polymer with a reactive ester group that is capable of reacting with the therapeutic agent. Examples of such compounds include N-hydroxysuccinimide, 4-hydroxy-3-nitrobenzene sulfonic acid, and the like, and preferably N-hydroxysuccinimide or 4-hydroxy-3-nitrobenzene sulfonic acid is used. For example, monomethyl substituted PEG may be reacted at elevated temperatures, preferably about 100-110° C. for four hours, with glutaric anhydride. The monomethyl PEG-glutaric acid thus produced is then reacted with N-hydroxysuccinimide in the presence of a carbodiimide reagent such as dicyclohexyl or isopropyl carbodiimide to produce the activated polymer, methoxypolyethylene glycolyl-N-succinimidyl glutarate, which can then be reacted with the therapeutic agent. This method is described in detail in Abuchowski et al., Cancer Biochem. Biophys. 7:175-186 (1984). In another example, the monomethyl substituted PEG may be reacted with glutaric anhydride followed by reaction with 4-hydroxy-3-nitrobenzene sulfonic acid (HNSA) in the presence of dicyclohexyl carbodiimide to produce the activated polymer. HNSA is described by Bhatnagar et al., Peptides: Synthesis-Structure-Function, Proceedings of the Seventh American Peptide Symposium, Rich et al. (eds.) (Pierce Chemical Co., Rockford Ill., 1981), p. 97-100, and in Nitecki et al., High-Technology Route to Virus Vaccines (American Society for Microbiology: 1986) entitled “Novel Agent for Coupling Synthetic Peptides to Carriers and Its Applications.”

Specific methods of producing therapeutic peptide conjugated to PEG include the methods described in U.S. Pat. No. 4,179,337 on PEG-therapeutic agent and U.S. Pat. No. 4,935,465, which discloses PEG reversibly but covalently linked to therapeutic peptides. Other specific methods for producing PEG-therapeutic agent include the following:

PEGylation with methoxypolyethylene glycol aldehyde (Me-PEG aldehyde) by reductive alkylation and purification is accomplished by adding to 2 mg/mL of therapeutic agent in PBS pH 7.0, 5 mM of Me-PEG aldehyde-5000 (molecular weight 5000 daltons) and 20 mM of NaCNBH3 and gently mixing at room temperature for 3 hours. Ethanolamine is then added to 50 mM to reductively amidate the remaining unreacted Me-PEG. The mixture is separated on an anion-exchange column, FPLC Mono Q. The surplus unreacted Me-PEG does not bind to the column and can then be separated from the mixture. Two main PEGylated therapeutic agent fractions are obtained with apparent molecular weights of 30K and 40K on reduced SDS-PAGE, vs. 20K of the unreacted therapeutic agent. therapeutic agent-GHBP complex is PEGylated in the same manner to give a derivative of 150K by gel filtration.

PEGylation with N-hydroxysuccinimidyl PEG (NHS-PEG) and purification are accomplished by adding NHS-PEG at a 5-fold molar excess of the total lysine concentration of therapeutic agent to a solution containing 2 mg/mL of therapeutic agent in 50 mM of sodium borate buffer at pH 8.5 or PBS at pH 7, and mixing at room temperature for one hour. Products are separated on a Superose 12 sizing column and/or Mono Q of FPLC. The PEGylated therapeutic agent varies in size depending on the pH of the reaction from approximately 300 K for the reaction run at pH 8.5 to 40 K for pH 7.0 as measured by gel filtration. The therapeutic agent-GHBP complex is also PEGylated the same way with a resulting molecular weight of 400 to 600 Kd from gel filtration.

PEGylation of the cysteine mutants of therapeutic peptides with PEG-maleimide is accomplished by preparing a single cysteine mutant of therapeutic peptide by site-directed mutagenesis, secreting it from an E. coli 16C9 strain (W3110 delta tonA phoA delta E15 delta (argF-lac) 169 deoC2 that does not produce the deoC protein and is described in U.S. Ser. No. 07/224,520 filed Jul. 26, 1988, now abandoned, the disclosure of which is incorporated herein by reference) and purifying it on an anion-exchange column. PEG-maleimide is made by reacting monomethoxyPEG amine with sulfo-MBs in 0.1 M sodium phosphate pH 7.5 for one hour at room temperature and buffer exchanged to phosphate buffer pH 6.2. Next therapeutic peptide with a free extra cysteine is mixed in for one hour and the final mixture is separated on a Mono Q column as in Me-PEG aldehyde PEGylated therapeutic peptide.

As ester bonds are chemically and physiologically labile, it may be preferable to use a PEG reagent in the conjugating reaction that does not contain ester functionality. For example, a carbamate linkage can be made by reacting PEG-monomethyl ether with phosgene to give the PEG-chloroformate. This reagent could then be used in the same manner as the NHS ester to functionalize lysine side-chain amines. In another example, a urea linkage is made by reacting an amino-PEG-monomethyl ether with phosgene. This would produce a PEG-isocyanate that will react with lysine amines.

A preferred manner of making PEG-therapeutic agent, which does not contain a cleavable ester in the PEG reagent, is described as follows: Methoxypoly(ethylene glycol) is converted to a carboxylic acid by titration with sodium naphthalene to generate the alkoxide, followed by treatment with bromoethyl acetate to form the ethyl ester, followed by hydrolysis to the corresponding carboxylic acid by treatment with sodium hydroxide and water, as reported by Buckmann et al., Macromol. Chem., 182:1379-1384 (1981). The resultant carboxylic acid is then converted to a PEG-N-hydroxysuccinimidyl ester suitable for acylation of therapeutic agent by reaction of the resultant carboxylic acid with dicyclohexylcarbodiimide and NHS in ethyl acetate.

The resultant NHS-PEG reagent is then reacted with 12 mg/mL of therapeutic agent using a 30-fold molar excess over therapeutic agent in a sodium borate buffer, pH 8.5, at room temperature for one hour and applied to a Q Sepharose column in Tris buffer and eluted with a salt gradient. Then it is applied to a second column (phenyl Toyopearl) equilibrated in 0.3 M sodium citrate buffer, pH 7.8. The PEGylated therapeutic agent is then eluted with a reverse salt gradient, pooled, and buffer-exchanged using a G25 desalting column into a mannitol, glycine, and sodium phosphate buffer at pH 7.4 to obtain a suitable formulated PEG7-therapeutic agent.

The PEGylated therapeutic agent molecules and therapeutic agent-GHBP complex can be characterized by SDS-PAGE, gel filtration, NMR, tryptic mapping, liquid chromatography-mass spectrophotometry, and in vitro biological assay. The extent of PEGylation is suitably first shown by SDS-PAGE and gel filtration and then analyzed by NMR, which has a specific resonance peak for the methylene hydrogens of PEG. The number of PEG groups on each molecule can be calculated from the NMR spectrum or mass spectrometry. Polyacrylamide gel electrophoresis in 10% SDS is appropriately run in 10 mM Tris-HCl pH 8.0, 100 mM NaCl as elution buffer. To demonstrate which residue is PEGylated, tryptic mapping can be performed. Thus, PEGylated therapeutic agent is digested with trypsin at the protein/enzyme ratio of 100 to 1 in mg basis at 37° C. for 4 hours in 100 mM sodium acetate, 10 mM Tris-HCl, 1 mM calcium chloride, pH 8.3, and acidified to pH<4 to stop digestion before separating on HPLC Nucleosil C-18 (4.6 mm.times.150 mm, 5μ, 100 A). The chromatogram is compared to that of non-PEGylated starting material. Each peak can then be analyzed by mass spectrometry to verify the size of the fragment in the peak. The fragment(s) that carried PEG groups are usually not retained on the HPLC column after injection and disappear from the chromatograph. Such disappearance from the chromatograph is an indication of PEGylation on that particular fragment that should contain at least one lysine residue. PEGylated therapeutic agent may then be assayed for its ability to bind to the GHBP by conventional methods.

The various PEGylation methods used produced various kinds of PEGylated wild-type therapeutic agent, with apparent molecular weights of 35K, 51K, 250K, and 300K by size exclusion chromatography, which should be close to their native hydrodynamic volume. These were designated PEG1-therapeutic agent, PEG2-therapeutic agent, PEG3-therapeutic agent, and PEG7-therapeutic agent, respectively. From the results of the tryptic mapping, the PEG1-therapeutic agent and PEG2-therapeutic agent both had the N-terminal 9-amino-acid fragment missing from the chromatogram and possibly PEGylated, which could be confirmed by the mass spectrometry of the big molecular species found in the flow-through of the liquid chromatograph. From the molecular weight on SDS-PAGE, PEG1-therapeutic agent may have one PEG on the N-terminal amine, and the PEG2-therapeutic agent may have two PEG molecules on the N-terminal amine, forming a tertiary amide. The PEG3-therapeutic agent has about 5 PEG groups per molecule based upon the NMR result, and on the tryptic map, at least five peptide fragments were missing, suggesting that they are PEGylated. The PEG7-therapeutic agent molecule is believed to have 6-7 PEG groups per molecule based on mass spectrometry.

### Diseases Amenable to Treatment with the Compositions and Methods of the Invention

The current broad indications for liver cell transplantation are usually accepted to be either an unacceptable quality of life (because of liver disease) or anticipated length of life is less than one year (because of liver disease). There are also different criteria for pediatric transplantation versus adult transplantation. The British Society of Gastroenterology has published guidelines on the indications for referral and assessment in adult liver transplantation: a clinical guide (Devlin J, O'Grady J, Gut 1999:45; suppl 6; vi1-vi22).

Some examples of patients that are amenable to treatment by the compositions and methods of the present invention are in the following table:

PRIMARY RECIPIENT DISEASE

| Cirrhosis: | Secondary sclerosing cholangitis |
|---|---|
| Primary biliary cirrhosis | Alpha -1-antitrypsin deficiency |
| Secondary biliary cirrhosis | Budd-Chiari syndrome |
| Cryptogenic | Wilson's disease |
| Alcoholic | Biliary atresia |
| Non-alcoholic steatotic | Other congenital biliary abnormalities |
| hepatitis | |
| Chronic active hepatitis | Acute/subacute fulminant hepatic failure |
| (autoimmune) | (FHF) |
| Chronic viral hepatitis B | Primary hepatocellular CA in cirrhotic |
| | liver |
| Chronic viral hepatitis C | Primary hepatic malignancy |
| Congenital hepatic fibrosis | Inborn errors of metabolism not in CLF |
| | group |
| Primary sclerosing cholangitis | |

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Clinical Investigations Using Relaxin H2 and Follistatin 315

The first clinical trial addresses non-alcoholic steatotic fibrosis. The dosage and delivery mechanism for relaxin H2 is subcutaneous delivery of the molecule based on the earlier reports for the scleroderma trial. Seibold J R et al., Ann Intern Med. 2000 Jun. 6; 132(11):871-9. The dosage of relaxin H2 in the present trial is the most efficacious level that was used in the earlier reported scleroderma trial, a subcutaneous relaxin infusion rate of 25 μg/kg/day, with the mode of delivery via continuous subcutaneous delivery (e.g., through implantation of an osmotic pump). The simultaneous dosage of follistatin will be 50 μg/kg/day through subcutaneous delivery.

Trial Protocol

A formulation comprised of rH2 relaxin and recombinant human follistatin 315 is used to improve prognosis and decrease on-going damage caused from on-going hepatic damage in patients diagnosed with moderately severe NASH. The measured end points are indicative of a decrease of reduction of area of damage in the liver and increased liver enzyme function.

The patients in the trial are randomized into the following four dosage groups:

1) Administration of rH2 relaxin alone (10-15 patients)

2) Administration of follistatin 315 alone (10-15 patients)

3) Administration of both rH2 relaxin and follistatin 315 alone (10-15 patients)

4) Administration of vehicle alone (10-15 patients)

Patients are randomly assigned to receive subcutaneous delivery of rH2 relaxin alone, subcutaneous delivery of a formulation comprising rH2 relaxin plus follistatin, or a subcutaneous delivery of vehicle alone. The groups receiving relaxin and follistatin show a significant improvement over those treated with vehicle, and patients with combination therapy showed increased efficacy over both forms of monotherapy.

Measurement of End Points

rH2 relaxin and follistatin administration are studied in a small patient population of NASH and an improvement in liver enzyme levels and liver function is measured as a primary end point. A decrease in on-going damage as determined by imaging techniques is also measured. Additional end points are selected to provide the maximum amount of relevant information within a reasonable timeframe. They include:
1. Assessment of hepatic fibrosis changes by measurement of area of fibrosis (image analysis method)
2. Assessment of hepatic fibrosis changes by blood scores of fibrosis (ALT, AST, alkaline phosphatase, direct and total bilirubin, and albumin); hyaluronate dosage; prothrombin time and alpha 2 macroglobulinemia dosages
3. Cytokine levels (i.e. TNF-α, IL-6, IL-10) and expression of TNF-α receptors (p55 and p75)
4. Liver histology following treatment using MRI and/or PET imaging.
   In addition, elements such as diet and exercise are carefully monitored in the trial populations as these may impact on the improvement of the underlying metabolic disorder and potentially skew the results.

While this invention is satisfied by embodiments in many different forms, as described in detail in connection with the various embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims filed in the corresponding utility application, unless the term “means” is used, none of the features or elements recited therein should be construed as means-plus-function limitations pursuant to 35 U.S.C. §112, ¶6.

What is claimed is:

1. A method of treating hepatic fibrosis, comprising the steps of:
(a) measuring a liver function parameter of a patient to determine a need for administration of the therapeutic agents;
(b) administering to a patient a dosage form comprising one or a combination of therapeutic agents;
(c) remeasuring the parameter of the patient measured in (a) after administration of the therapeutic agents; and
(d) readministering the formulation to the patient.

2. The method of claim 1, wherein the dosage form administration in step (b) is administered as a single dosage.

3. The method of claim 1, wherein the dosage form administration in step (d) is administered as a single dosage.

4. The method of claim 1, wherein the dosage form administration in step (b) is administered in multiple dosages.

5. The method of claim 1, wherein the dosage form administration in step (d) is administered in multiple dosages.

6. The method of claim 1, wherein the dosage form administered in steps (b) and (d) comprises at least two therapeutic agents.

7. The method of claim 6, wherein the therapeutic agents are administered separately.

8. The method of claim 1, wherein the liver function parameter measured is area of fibrosis as determined by image analysis.

9. The method of claim 8, wherein the image analysis is determined using magnetic resonance imaging (MRI) or Positron Emission Tomography (PET).

10. The method of claim 1, wherein the liver function parameter is a blood score of fibrosis.

11. The method of claim 1, wherein the blood score of fibrosis measures a change in ALT, AST, alkaline phosphatase, direct and total bilirubin, or albumin.

12. The method of claim 1, wherein the liver function parameter is a change in liver histology as determined through liver biopsy.

13. The method of claim 1, wherein the therapeutic agent is administered through intravenous injection.

14. The method of claim 1, the therapeutic agent is administered through intraportal injection.

15. A method of treating hepatic fibrosis, comprising the steps of:
(a) measuring a liver function parameter of a patient to determine a need for administration of the therapeutic agents;
(b) administering to a patient a formulation comprising one or a combination of therapeutic agents;
(c) remeasuring the parameter of the patient measured in (a) after administration of the therapeutic agents;
(d) readministering the formulation to the patient;
(e) adjusting the amount of formulation readministered in (d) based on an analysis of results obtained in remeasuring in (c).

16. The method of claim 15, wherein the invention further provides the step of: repeating (a)-(e) a plurality of times.

17. The method of claim 16, wherein steps (a)-(e) are repeated until a measured parameter is improved by 10% or more relative to a level measured in the first step (a).

18. The method of claim 15, wherein the formulation is administered through intravenous injection.

19. The method of claim 15, the formulation is administered through intraportal injection.
